(19) United States
(12) Patent Application Publication
(10) Pub. No.: US 2017/0326379 A1
(43) Pub. Date: Nov. 16, 2017

(54) OPTICAL CONTROL SYSTEM FOR LOWER URINARY TRACT DYSFUNCTIONS

(71) Applicant: KOREA INSTITUTE OF SCIENCE AND TECHNOLOGY, Seoul (KR)

(72) Inventors: Jun-Kyo Francis SUH, Seoul (KR); Jae Hong PARK, Seoul (KR); Hyun Joon SHIN, Seoul (KR); Jinki HONG, Seoul (KR)

(73) Assignee: KOREA INSTITUTE OF SCIENCE AND TECHNOLOGY, Seoul (KR)

(21) Appl. No.: 15/276,923

(22) Filed: Sep. 27, 2016

(30) Foreign Application Priority Data
May 16, 2016 (KR) 10-2016-0059643

(51) Int. Cl. A61N 5/06; A61B 8/08; A61K 41/00

(52) U.S. Cl. CPC A61N 5/062; A61K 41/0057; A61N 5/0601; A61B 8/48; A61N 2005/061; A61N 2005/0645; A61N 2005/0654; A61N 2005/0663; A61N 2005/0626

(57) ABSTRACT

A urinary control system for controlling urination has a first light source configured to selectively apply a first light and a second light to a bladder muscle and a second light source configured to selectively apply the first light and the second light to the urethral sphincter. A first optogene is expressed to the bladder muscle and the urethral sphincter, and a second optogene is expressed to the bladder muscle and the urethral sphincter, the first optogene contracts muscles by depolarizing membrane potential when the first light is applied, the second optogene relaxes muscles by depolarizing membrane potential when the second light is applied, and the contraction and relaxation of the bladder muscle and the urethral sphincter are achieved counteractively via photostimulation by the first light and the second light, respectively, so as to control urination.

### CROSS-REFERENCE TO RELATED APPLICATION

This application claims the priority of Korean Patent Application No. 10-2016-0059643, filed on May 16, 2016, and all the benefits accruing therefrom under 35 U.S.C. §119, the contents of which in its entirety are herein incorporated by reference.

### BACKGROUND

### 1. Field

The present disclosure relates to a urinary control system utilizing an optogenetic system, more specifically to a urinary control system for actively controlling the urination-associated muscle of a patient suffering from lower urinary tract dysfunction using photostimulation to allow for urination to the level of normal people.

[Description about National Research and Development Support]

This study was supported by the National Agenda Project of Ministry of Science, ICT and Future Planning, Republic of Korea (Project No. NAP-09-4-KIST) under the superintendence of National Research Council of Science & Technology of Korea.

### 2. Description of the Related Art

Urination is an important metabolic process for expelling urinary waste materials out of the body. Urine is produced by the kidney and stored in the bladder. The bladder stores the urine flowing from the kidney and releases it out of the body at once at a proper time.

FIG. 1 and FIG. 2 describe the process of urination. FIG. 1 shows a state where urine is stored in the bladder and FIG. 2 shows a state where the urine is excreted.

The bladder 1 is increased in volume when urine is stored due to its contraction/relaxation property. The bladder wall is composed of thick bladder muscles 10. Because collagen surrounds muscle cells like nets, the bladder muscle 10 is elastic and tough.

Neurites 11 emanating from the spine are located in the bladder muscles 10 and, when the bladder is filled with urine, send a signal that the bladder 1 is filled with urine to the brain. Then, when the brain sends a voluntary signal, the bladder 1 contracts.

The urethra 2 is connected below the bladder 1. The urethra 2 has the urethral sphincter 20, which constricts the urethra when urine is filled in the bladder. The urethral sphincter 20 is also composed of contracting/relaxing tissue and its contraction/relaxation is controlled by neurites 21.

When a predetermined amount (usually about 400 mL) of urine is filled in the bladder 1, the bladder 1 sends a signal informing the situation to the brain via the spinal cord. The brain then sends a command signal of urination at a proper time to the bladder 1 via the spinal cord. Then, urine is excreted out of the body as the bladder muscle 10 contracts and at the same time the urethral sphincter 20 relaxes.

This urination process is primarily undertaken by the bladder and the urethra but is elaborately controlled by the brain, the spinal cord and peripheral nerves.

Denaturation of the bladder muscle, interruption of nerves connected to the bladder, etc. may cause problems in urinary control.

This urinary control disorder may cause lower urinary tract symptoms, urinary incontinence, overactive bladder syndrome, underactive bladder syndrome, etc.

There have been attempts to control the bladder muscle through medication or electrical stimulation in order to solve the urinary control disorder. However, medication has the problem that the brain or other organs are also affected because the drug targets not only the urination-related tissues but also other tissues. As a result, side effects such as mouth dryness, constipation, abnormalities of the heartbeat, etc. may occur. For electrical stimulation, it is very difficult to target specific cells only because all biological tissues in living body contain electrolytes, thus electrically conductive. Because the urinary nervous system is composed together with the nerves associated with defecation and sexual function, it is very difficult to specifically control urination only through electrical stimulation.

### SUMMARY

The present disclosure is directed to providing a urinary control system which is capable of effectively controlling regulation by actively controlling the contraction and relaxation of muscles associated with urination using photostimulation.

In an aspect, the present disclosure provides a urinary control system for controlling urination by controlling the action of muscles associated with urination including a first light source configured to selectively emit a first light and a second light to a bladder muscle and a second light source configured to selectively emit the first light and the second light to the urethral sphincter, wherein a first optogene is expressed to the bladder muscle and the urethral sphincter, and a second optogene is expressed to the bladder muscle and the urethral sphincter, the first optogene contracts muscles by depolarizing membrane potential when the first light is applied, the second optogene relaxes muscles by depolarizing membrane potential when the second light is applied, and the contraction and relaxation of the bladder muscle and the urethral sphincter are achieved counteractively via photostimulation by the first light and the second light, respectively, so as to control urination.

In an exemplary embodiment, the first light source is a lamp which illuminates the first and second lights inside the bladder toward the inner wall of the bladder in all directions.

In an exemplary embodiment, the second light source is a cuff-type lamp around the urethral sphincter which illuminates the first and second lights onto the urethral sphincter.

In an exemplary embodiment, the first light source and the second light source are lamps which are inserted in the outer membrane of muscles.

In an exemplary embodiment, the first light source and the second light source are attached to a waist-belt and illuminate light into the body from outside.

In an exemplary embodiment, the urinary control system further includes a urine volume sensor which detects the urine volume contained in the bladder.

In an exemplary embodiment, the urine volume sensor estimates the volume of the urine in the bladder using an acoustic signal reflected when an ultrasonic wave from the sensor is emitted into the bladder.

In an exemplary embodiment, the urine volume sensor also provides the information about the change rate of urine volume in the bladder.

In an exemplary embodiment, the first optogene can be channelrhodopsin-2 and the first light (e.g. blue light) can be used to activate channelrhodopsin-2. The second optogene can be halorhodopsin, and the second light (e.g. yellow light) can be used to activate halorhodopsin.

### BRIEF DESCRIPTION OF THE DRAWINGS

FIG. 1 and FIG. 2 describe the process of urination from a normal bladder.

FIG. 3 describes the expression of optogenes in the bladder muscle and the urethral sphincter for restoration of urinary function.

FIG. 4A and FIG. 4B show the depolarization of membrane potential of channelrhodopsin-2 expressing bladder muscle cell under blue-light illumination.

FIGS. 5A-5C show contraction pressure of bladder when blue-light photostimulation is applied to a channelrhodopsin-2 expressing bladder muscle.

FIG. 6A and FIG. 6B show the hyperpolarization of membrane potential of halorhodopsin-expressing bladder muscle cell under yellow-light illumination.

FIG. 7 shows that an overactive bladder contraction induced by prostaglandin E2 (PGE2) treatment was inhibited by application of yellow-light photostimulation to halorhodopsin-expressing bladder muscle.

FIG. 8A and FIG. 8B describe the control of urination using a urinary control system according to an exemplary embodiment of the present disclosure.

FIGS. 9-11 show various exemplary embodiments of a first light source and a second light source.

FIG. 12 shows a urine volume sensor applied to the bladder.

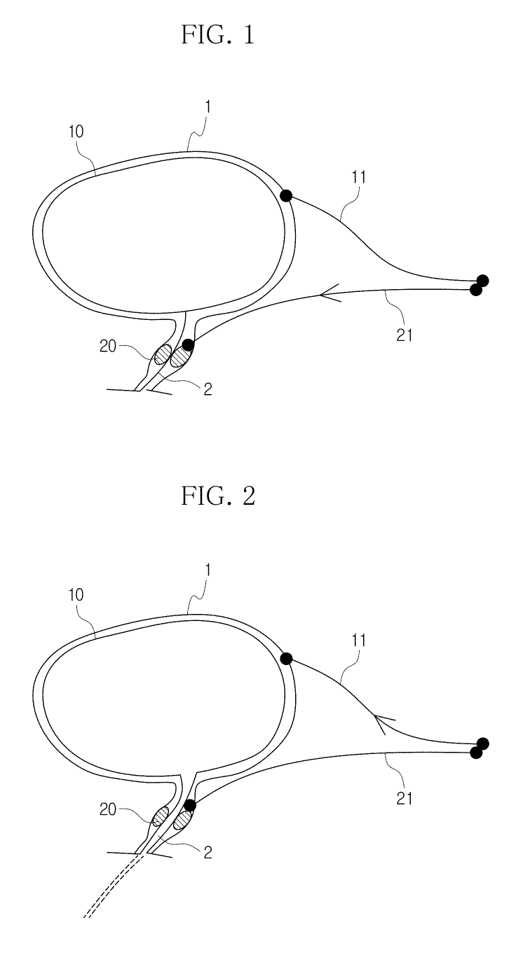

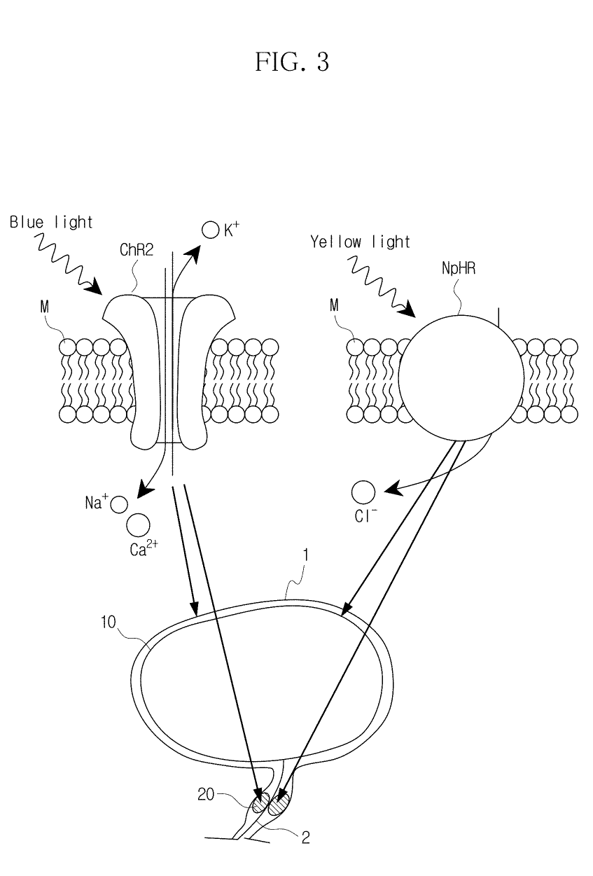

### DETAILED DESCRIPTION

Hereinafter, specific exemplary embodiments of the present disclosure are described with reference to the accompanying drawings. Although the present disclosure is described referring to the exemplary embodiments shown in the drawings, the scope of the present disclosure is not limited by the exemplary embodiments.

A urinary control system according to an exemplary embodiment of the present disclosure controls urination by stimulating the bladder muscle 10 and the urethral sphincter 20 which store and release urine through counteracting contraction/relaxation mechanisms.

For this, in an exemplary embodiment, urination is controlled using optogenetic photostimulation which is advantageous over other conventional treatment methods, such as drug therapy and electrical stimulation, in terms of biocompatibility and spatiotemporal resolution. Because the bladder muscle and the urethral sphincter are not photoresponsive, in an exemplary embodiment, optogenetic technique is used to confer photoresponsiveness.

Specifically, in an exemplary embodiment, optogenes that can induce the change in membrane potential by photostimulation are introduced and expressed in the bladder muscle 10 and the urethral sphincter 20. This will allow the otherwise photo-insensitive bladder muscle 10 and the urethral sphincter 20 to become photo-sensitive and thereby to contract and relax by photostimulation.

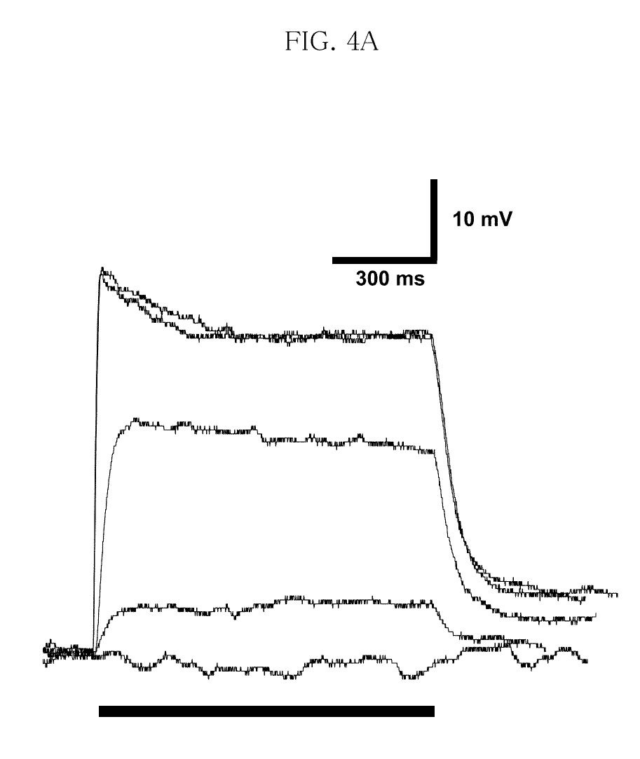

FIG. 3 describes the expression of optogenes in the bladder muscle 10 and the urethral sphincter 20 for restoration of urinary function.

In an exemplary embodiment, the optogene is channelrhodopsin-2 (ChR2) and/or halorhodopsin (NpHR).

Channelrhodopsin-2 is a cation (Na⁺, K⁺, Ca²⁺) channel membrane protein extracted from algae (Chlamydomonas reinhardtii) and can be activated by blue-light photostimulation (473 nm). Specifically, when channelrhodopsin-2 ion channel is activated by blue light, Na⁺ and Ca²⁺ enter into the cell through the activated channel, thereby depolarizing the cell membrane. As a result, the action potentials of nerve cells and muscle cells are triggered.

Halorhodopsin is a chloride ion (Cl⁻)-pumping membrane protein, which can be activated by yellow light (593 nm). Specifically, halorhodopsin, when activated by yellow light, transports chloride ion (Cl⁻) from the extracellular domain into the intracellular domain, thereby inducing hyperpolarization of membrane potential. As a result, the action potentials of nerve cells and muscle cells are suppressed.

The optogenes such as channelrhodopsin-2 and halorhodopsin may be selectively delivered into the cell for their expressions in the cell membrane using various known methods.

In an exemplary embodiment, the channelrhodopsin-2 and halorhodopsin genes may be specifically inserted into the bladder muscle and the urethral sphincter using viral gene transfection methods which utilize adeno- or adeno associate-viruses or non-viral gene transfection methods which utilize lipofectamine or polymeric nano-particles, etc.

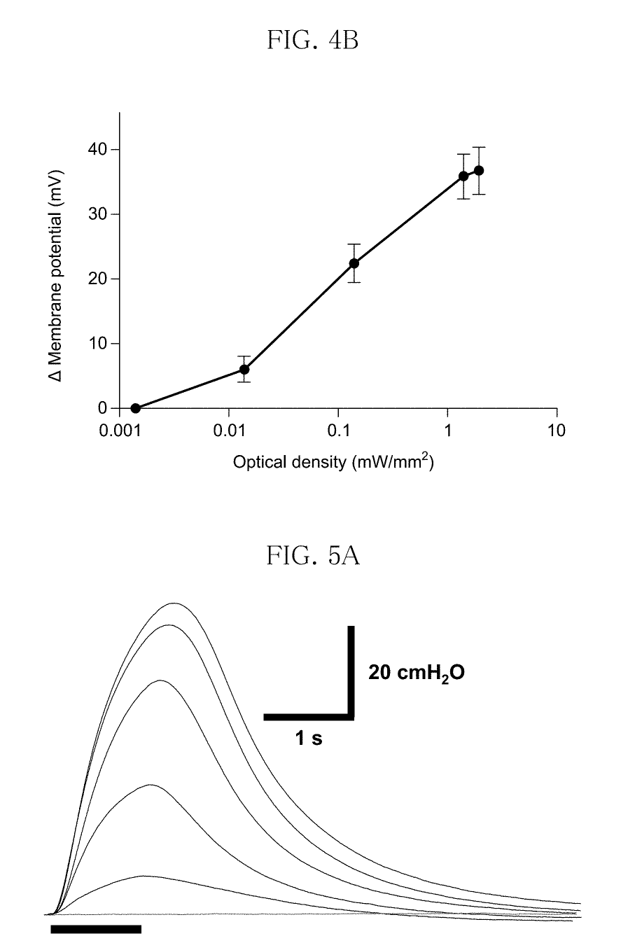

FIG. 4A and FIG. 4B show the depolarization of membrane potential occurring when blue-light photostimulation is applied to a bladder muscle cell.

In FIG. 4A, the abscissa indicates time and the thick line below the graph indicates the time duration during which blue-light photostimulation was applied. In FIG. 4A, the ordinate indicates the induced membrane potential. From FIG. 4A, it can be seen that the membrane potential of the bladder muscle cell is depolarized during the blue-light photostimulation.

Also, from FIG. 4A and FIG. 4B, it can be seen that the magnitude of the membrane potential induced by photostimulation increases with optical density.

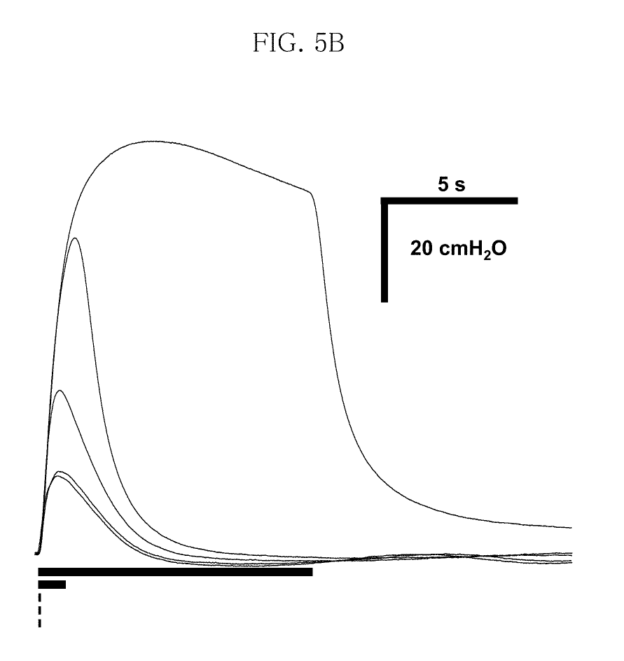

FIGS. 5A-5C show the pressure of the bladder 1 when blue-light photostimulation is applied.

In FIG. 5A and FIG. 5B, the thick line below the abscissa indicates the time duration during which blue-light photostimulation was applied. From FIG. 5A and FIG. 5B, it can be seen that bladder pressure is increased by blue-light photostimulation and the bladder pressure increases further as the photostimulation time is longer. In addition, it can be seen from FIG. 5C that the bladder pressure increases with the intensity of the light.

The increase in the bladder pressure means that the bladder contracts. Accordingly, it was confirmed that, due to the action of channelrhodopsin-2 by the blue-light photostimulation, the membrane potential of the bladder muscle cell is depolarized and the contraction of the bladder can be induced.

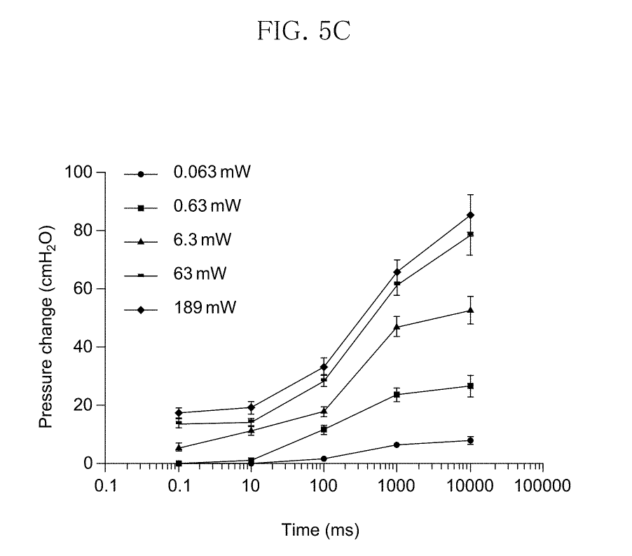

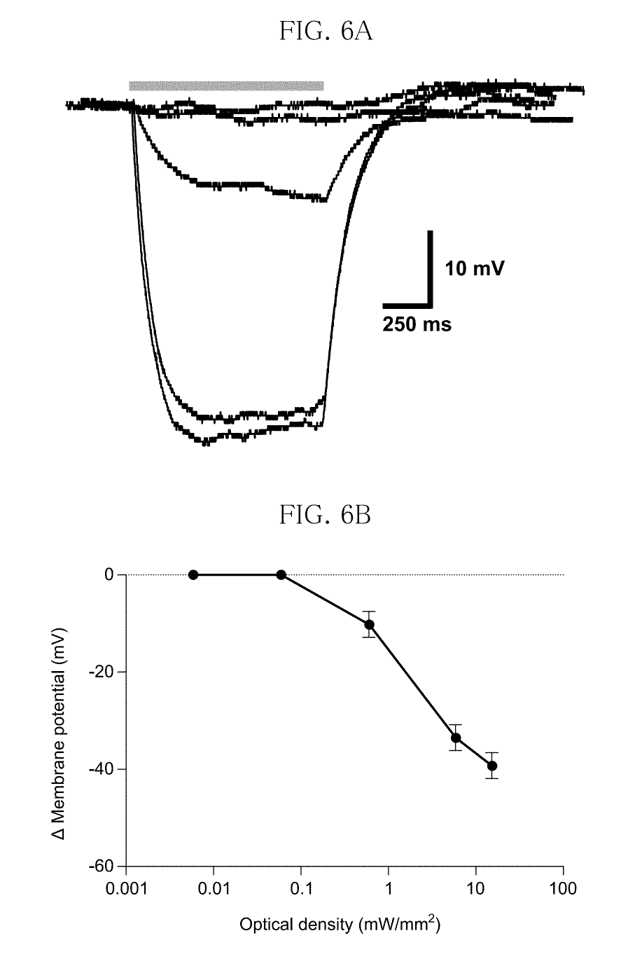

FIGS. 6 to 7 show the result for halorhodopsin. Yellow-light photostimulation was applied to halorhodopsin-expressing bladder muscle.

FIG. 6A and FIG. 6B show the hyperpolarization of membrane potential occurring when yellow-light photostimulation is applied to the bladder muscle cell.

In FIG. 6A, the abscissa indicates time and the thick line above the graph indicates the time duration during which yellow-light photostimulation was applied. In FIG. 6A, the ordinate indicates the membrane potential induced by the yellow light. From FIG. 6A, it can be seen that the membrane potential of the bladder muscle cell is hyperpolarized during the yellow-light photostimulation. Also, from FIG. 6A and FIG. 6B, it can be seen that the magnitude of the absolute value of the hyperpolarized membrane potential induced by photostimulation increases with optical density.

FIG. 7 shows the inhibition of overactive bladder induced by prostaglandin E2 (PGE2) (the thick line below the graph indicates the time duration during which the bladder sample was treated with PGE2). Overactive bladder contraction can be seen as the result of PGE2 treatment.

From FIG. 7, it can be seen that the overactive bladder syndrome induced by PGE2 is alleviated during the yellow-light application (indicated as the gray line in the graph) and this phenomenon is reversible and repeatable.

Accordingly, it was confirmed that the activation of halorhodopsin by the yellow-light photostimulation leads to the hyperpolarization of the membrane potential of the bladder muscle cell and induces bladder relaxation.

Since the urethral sphincter is also a contracting/relaxing tissue like the bladder muscle, its contraction/relaxation can be achievable in the same way as the bladder muscle.

Referring again to FIG. 3, in an exemplary embodiment, both optogenes, i.e., channelrhodopsin-2 and halorhodopsin, can be expressed in the bladder muscle 10 and the urethral sphincter 20.

When contraction and relaxation of the bladder muscle 10 and the urethral sphincter 20 is necessary, their contraction/relaxation is precisely controlled spatiotemporally by illuminating blue and yellow light, respectively.

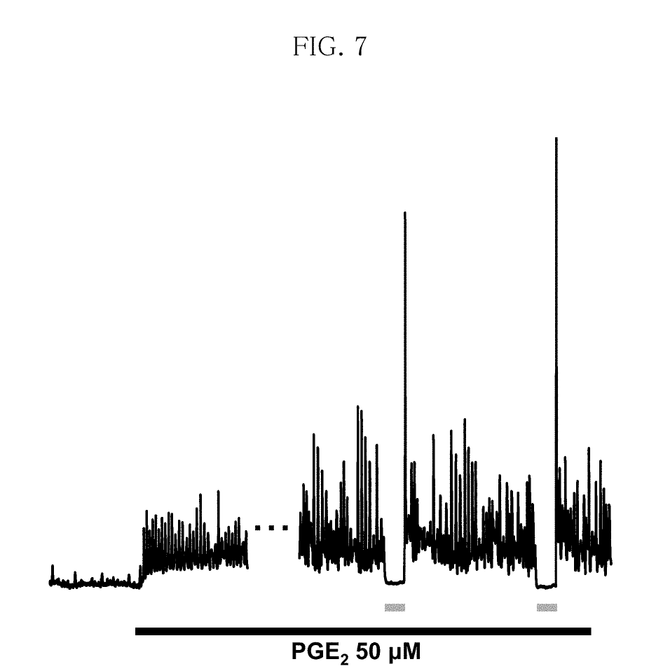

FIG. 8A and FIG. 8B describe the control of the bladder muscle and the urethral sphincter using a urinary control system according to an exemplary embodiment of the present disclosure.

The urinary control system includes a first light source 100 which is capable of selectively radiating a first light (blue light) 101 and a second light (yellow light) 102 and a second light source 200 which is capable of selectively radiating a first light (blue light) 201 and a second light (yellow light) 202.

The first light source 100 is arranged to apply light to the bladder muscle 10 and the second light source 200 is arranged to apply light to the urethral sphincter 20.

FIG. 8A describes the control of the discharge of urine.

As seen from FIG. 8A, in order to discharge urine through the urethra 2 out of the bladder 1, the first light source 100 applies blue light 101 to the bladder muscle 10 and the second light source 200 applies yellow light 202 to the urethral sphincter 20. As a result, urine is discharged from the bladder because the bladder muscle 10 contracts and the urethral sphincter 20 relaxes.

FIG. 8B describes the control of the storage of urine.

Oppositely from the above, in order to store urine in the bladder 1, the first light source 100 applies yellow light 102 to the bladder muscle 10 and the second light source 200 applies blue light 201 to the urethral sphincter 20. As a result, urine is stored because the bladder muscle relaxes and the urethral sphincter contracts.

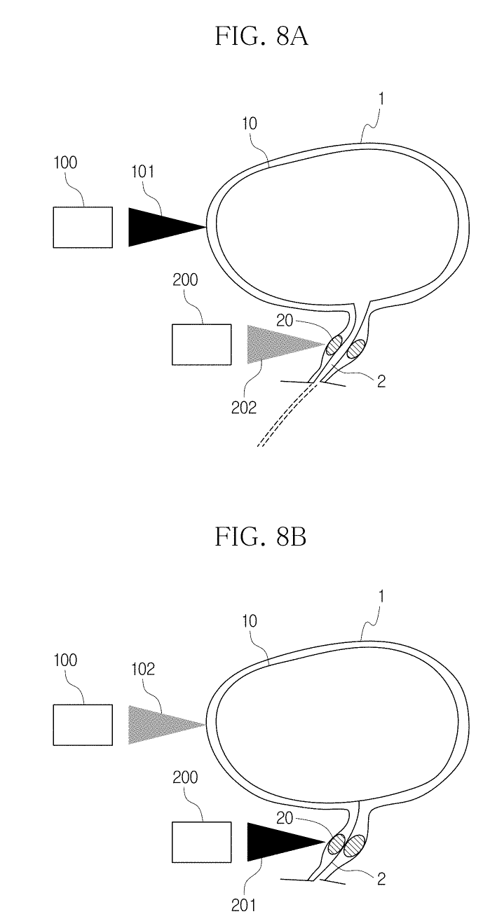

FIGS. 9-11 show various exemplary embodiments of the first light source 100 and the second light source 200.

As seen from FIG. 9, in an exemplary embodiment, the first light source 100 may be an LD, LED or OLED lamp inserted in the bladder 1. In an exemplary embodiment, the first light source 100 illuminates light L inside the bladder 1 toward the inner wall of the bladder in all directions.

Also, in an exemplary embodiment, the second light source 200 may be a cuff-type lamp which wraps around the urethral sphincter 20.

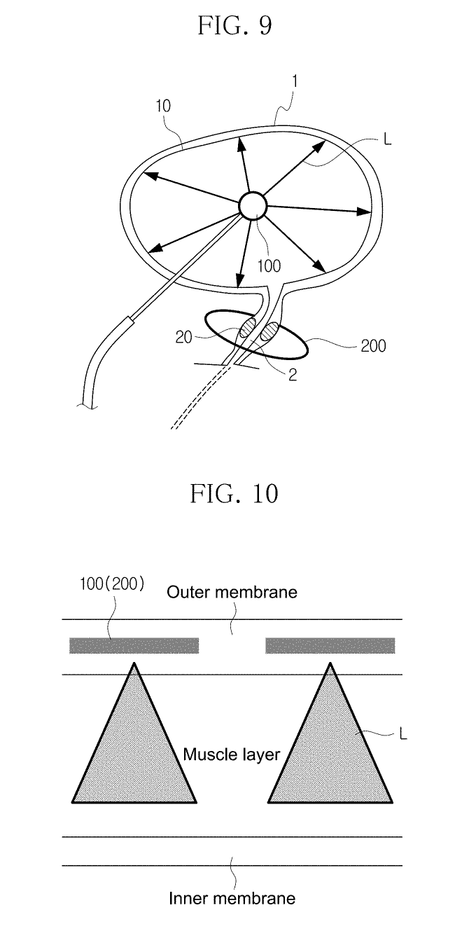

Alternatively, as seen from FIG. 10, the first light source 100 and the second light source 200 may be lamps which are inserted in the outer membrane of muscles (the bladder muscle and the urethral sphincter). This type of light source can apply more intense photostimulation to the targeted muscle cell in which the optogene is expressed and the target area or intensity of the photostimulation can be controlled more precisely by controlling each lamp independently, although a procedure of inserting it into the body is necessary.

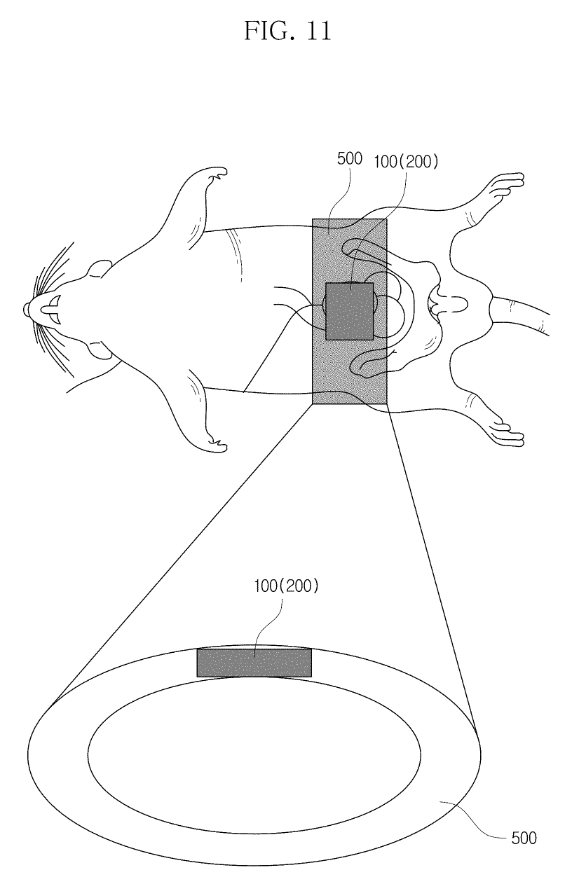

Alternatively, as seen from FIG. 11, the first light source 100 and the second light source 200 may be attached to a waist-belt 500 and illuminate light into the body from outside. In this case, the procedure of inserting a foreign body into the body is unnecessary.

In consideration of the time period required for urine being stored in the bladder 1 or being discharged out of the bladder 1, the storage control of urine (FIG. 8B) may need to be maintained for about 3 hours, whereas the discharge control (FIG. 8A) may need to be maintained for about 20-60 seconds.

For this, the urinary control system may be equipped with a timer for the discharge and storage control.

However, because the discharge of urine is not necessarily proportional to control time, a urinary control system according to another exemplary embodiment may further include a urine volume sensor which measures the volume of urine present in the bladder 1.

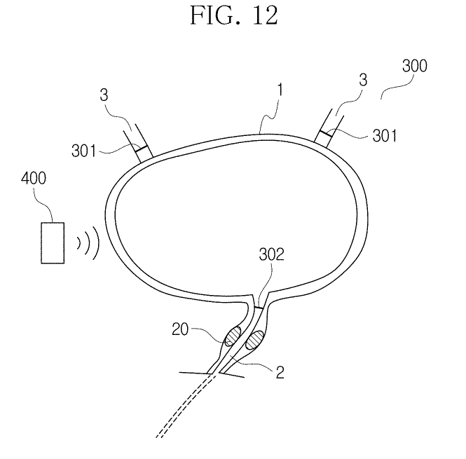

FIG. 12 shows a urine volume sensor applied to the bladder.

Referring to FIG. 12, the urine volume sensor is an acoustic sensor 400 which estimates the volume of urine present in the bladder using sound waves.

The sensor 400 estimates the amount of urine in the bladder from an acoustic signal reflected when ultrasonic waves are radiated to the bladder 1 from outside. This can be compared to the estimation of the state inside a barrel by tapping it and listening to the sound.

In another exemplary embodiment, the urine volume sensor may be a flow sensor 300 using a flow meter 301 provided at the ureter 3 and a flow meter 302 provided at the urethra 2.

The amount of urine stored in the bladder 1 can be calculated from the amount of urine flowing in thorough the ureter 3 and the amount of urine flowing out through the urethra 2.

The urinary control system according to the present disclosure allows for normal urination by controlling the storage and release of urine by controlling the light sources based on the volume of urine in the bladder measured by the residual urine sensor.

What is claimed is:

1. A urinary control system for controlling urination by controlling the action of muscles associated with urination, including:
a first light source configured to selectively emit a first light and a second light to a bladder muscle; and
a second light source configured to selectively emit the first light and the second light to the urethral sphincter,
wherein a first optogene is expressed to the bladder muscle and the urethral sphincter and a second optogene is expressed to the bladder muscle and the urethral sphincter,
the first optogene contracts muscles by depolarizing membrane potential when the first light is applied,
the second optogene relaxes muscles by depolarizing membrane potential when the second light is applied, and
the contraction and relaxation of the bladder muscle and the urethral sphincter are achieved counteractively via photostimulation by the first and second light, respectively, so as to control urination.

2. The urinary control system according to claim 1, wherein the first light source is a lamp which illuminates the first and second lights inside the bladder toward the inner wall of the bladder in all directions.

3. The urinary control system according to claim 1, wherein the second light source is a cuff-type lamp around the urethral sphincter which illuminates the first and second lights onto the urethral sphincter.

4. The urinary control system according to claim 1, wherein the first light source and the second light source are lamps which are inserted in the outer membrane of muscles.

5. The urinary control system according to claim 1, wherein the first light source and the second light source are attached to a waist-belt and illuminate light into the body from outside.

6. The urinary control system according to claim 1, which further comprises a urine volume sensor which measures the volume of urine present in the bladder.

7. The urinary control system according to claim 6, wherein the urine volume sensor estimates the change rate of urine volume from an acoustic signal reflected when ultrasonic waves are radiated to the bladder.

8. The urinary control system according to claim 1, wherein the first optogene is channelrhodopsin-2 and the second optogene is halorhodopsin.
